(19) United States
(12) Patent Application Publication
(10) Pub. No.: US 2011/0021877 A1
(43) Pub. Date: Jan. 27, 2011

(54) SURGICAL PORT AND FRANGIBLE INTRODUCER ASSEMBLY

(75) Inventors: Richard C. Fortier, Concord, MA (US); Jeffrey E. Ransden, Fairfield, CT (US); Leland R. Adams, Ansonia, CT (US); Joel N. Helfer, Cheshire, CT (US); Alan B. Bachman, Milford, MI (US); Adam I. Lehman, Northford, CT (US)

Correspondence Address: Tyco Healthcare Group LP;d/b/a Covidien, 555 Long Wharf Drive, Mail Stop 8-N1, Legal Department, New Haven, CT, 06511, US

(73) Assignee: TYCO Healthcare Group LP

(21) Appl. No.: 12/774,183

(22) Filed: May 5, 2010

Related U.S. Application Data
(60) Provisional application No. 61/228,204, filed on Jul. 24, 2009.

(51) Int. Cl. A61B 1/32

(52) U.S. Cl. 600/206

(57) ABSTRACT

A surgical portal and introducer assembly includes an introducer dimensioned for at least partial positioning within a tissue tract, and having a longitudinal introducer channel extending therethrough and a portal positionable within the longitudinal channel of the introducer. The introducer defines leading and trailing ends, and further has a frangible segment adapted to separate to expose the introducer channel. The portal has at least one longitudinal port for passage of a surgical object. The portal comprises a compressible material and is adapted to transition from a first expanded condition to a second compressed condition upon advancement through the longitudinal channel of the introducer to facilitate passage through the introducer whereby, upon separating of the frangible segment, the portal is released from the longitudinal channel to transition toward the first expanded condition to be generally secured within the tissue tract.

### CROSS REFERENCE TO RELATED APPLICATION

The present application claims the benefit of and priority to U.S. Provisional Application Ser. No. 61/228,204 filed on Jul. 24, 2009, the entire contents of which are incorporated herein by reference.

### BACKGROUND

1. Technical Field

The present disclosure relates generally to ports for use in minimally invasive surgical procedures, such as endoscopic and/or laparoscopic procedures, and more particularly, relates to an access port and an associated introducer to assist in deploying the port within a tissue tract of a patient.

2. Description of Related Art

Minimally invasive surgery is a type of surgery performed through one or more small incisions in a patient's body, usually less than an inch in diameter. Some potential advantages of minimal invasive surgery is that patients have less trauma to the body, lose less blood, have smaller surgical scars, and need less pain medication.

During a typical minimally invasive procedure, surgical objects, such as surgical access devices, e.g., trocar and cannula assemblies, or endoscopes, are inserted into the patient's body through the incision in tissue. In general, prior to the introduction of the surgical object into the patient's body, insufflation gas are used to enlarge the area surrounding the target surgical site to create a larger, more accessible work area. Accordingly, the maintenance of a substantially fluid-tight seal is desirable so as to prevent the escape of the insufflation gases and the deflation or collapse of the enlarged surgical site.

To this end, various ports with valves and seals are used during the course of minimally invasive procedures and are widely known in the art. However, a continuing need exists for an access port and associated introducer, which can position the access port with relative ease and with minor inconvenience for the surgeon.

### SUMMARY

Accordingly, a surgical portal and introducer assembly includes an introducer dimensioned for at least partial positioning within a tissue tract, and having a longitudinal introducer channel extending therethrough and a portal positionable within the longitudinal channel of the introducer. The introducer defines leading and trailing ends, and further has a frangible segment adapted to separate to expose the introducer channel. The portal has at least one longitudinal port for passage of a surgical object. The portal comprises a compressible material and is adapted to transition from a first expanded condition to a second compressed condition upon advancement through the longitudinal channel of the introducer to facilitate passage through the introducer whereby, upon separating of the frangible segment, the portal is released from the longitudinal channel to transition toward the first expanded condition to be generally secured within the tissue tract.

The frangible segment may include a tear line defined along the wall of the introducer. A tether may be secured to the introducer adjacent the tear line with the tether being manipulated to cause tearing along the tear line. The introducer may define a generally tapered configuration where an internal dimension of the longitudinal introducer channel generally decreases from the trailing end thereof to the leading end thereof.

The portal may define leading and trailing ends. The at least one longitudinal port may extend between the leading and trailing ends and is adapted for reception of an object whereby compressible material defining the at least one port is adapted to deform to establish a substantial sealed relation with the object. The portal may include a plurality of longitudinal ports. The portal may comprise one of a foam material or a gel material.

### DESCRIPTION OF THE DRAWINGS

The above and other aspects, features, and advantages of the present disclosure will become more apparent in light of the following detailed description when taken in conjunction with the accompanying drawings in which:

FIG. 1 is a side elevational view of the surgical portal and introducer assembly in accordance with the principles of the present disclosure;

FIG. 2 is a side elevational view of the surgical portal of the assembly of FIG. 1;

FIG. 3 is a side elevational view of the introducer of the assembly of FIG. 1; and

FIG. 4 is a partial cross-sectional view of another embodiment of the assembly.

### DETAILED DESCRIPTION

Particular embodiments of the present disclosure will be described herein with reference to the accompanying drawings. As shown in the drawings and as described throughout the following description, and as is traditional when referring to relative positioning on an object, the term “proximal” or “trailing” refers to the end of the apparatus that is closer to the user and the term “distal” or “leading” refers to the end of the apparatus that is further from the user. In the following description, well-known functions or constructions are not described in detail to avoid obscuring the present disclosure in unnecessary detail.

One type of minimal invasive surgery described herein is referred to as a single-incision laparoscopic surgery (SILS). SILS is an advanced minimally invasive surgical procedure, which would permit a surgeon to operate through a single entry point, typically the patient's navel. The disclosed SILS procedure involves insufflating the body cavity and positioning a portal member within, e.g., the navel of the patient. Instruments including an endoscope and additional instruments such as graspers, staplers, forceps or the like may be introduced within the portal member to carry out the surgical procedure.

The port assembly in the SILS procedure may be introduced into an incision with a Kelly clamp. However, the Kelly clamp may limit the surgeon's ability to properly place a SILS port due to the limited length of the Kelly clamp's arm and handle. Furthermore, visibility may become an issue due to the presence of the clamp and the surgeon's hand holding the clamp. Removal of the Kelly clamp subsequent to placement of the port may also present undesired obstacles.

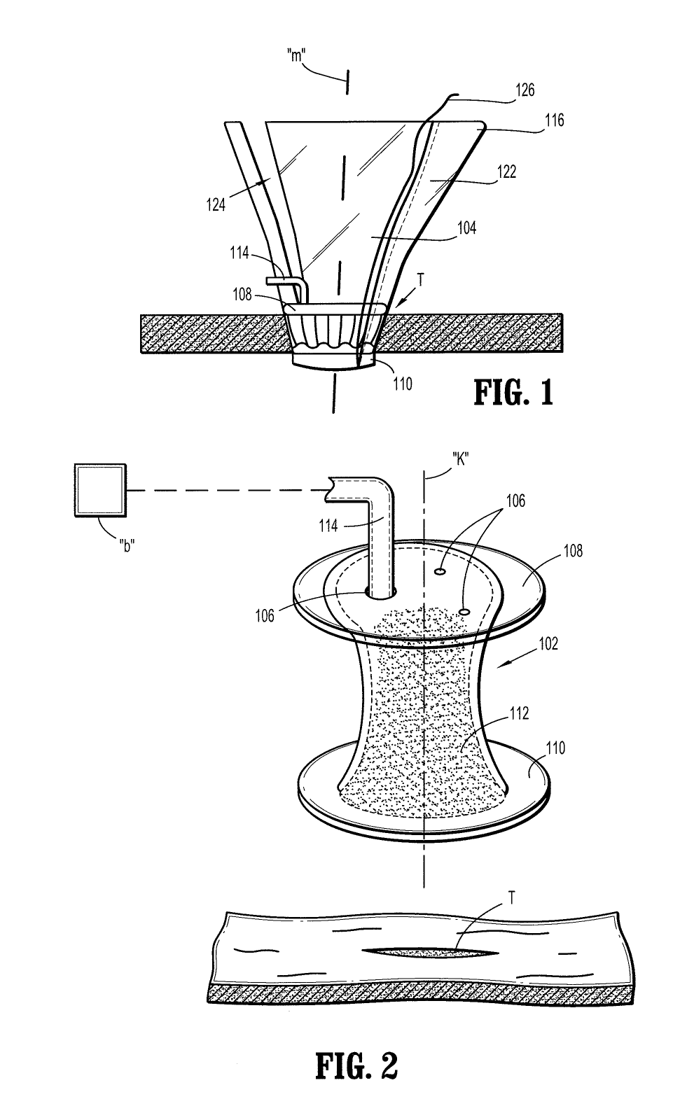

Referring now to the drawings, in which like reference numerals identify identical or substantially similar parts throughout the several views, FIG. 1 illustrates a surgical port and introducer assembly 100 including portal member 102 and introducer member 104 in accordance with the principles of the present disclosure. Portal member 102 is depicted enclosed within introducer member 104 in FIG. 1. As best depicted in FIG. 2, portal member 102 is adapted for insertion within a tissue tract “T”, e.g., through the abdominal or peritoneal lining in connection with a laparoscopic surgical procedure. Portal member 102 includes at least one longitudinal port 106, possibly, a plurality of longitudinal ports 106 extending along the axis “k” of the portal member 102. At least one or more inner longitudinal ports 106 are dimensioned to receive a surgical object, e.g. a surgical instrument (not shown) therethrough. Upon introduction through a respective port 106, the inner surface portions defining the port 106 establish and maintain a substantial sealed relation about the instrument or surgical object. Portal member 102 may define an hourglass shape as shown. Trailing and leading ends 108, 110 may define flange segments, which may be integrally formed with portal member 102. Portal member 102 may be made from a disposable, compressible, and/or flexible type material, for example, but not limited to, a suitable foam or gel material having sufficient compliance to form a seal about one or more surgical objects, and also establish a sealing relation with the tissue. The foam is preferably sufficiently compliant to accommodate off axis motion of the surgical object. In one embodiment, the foam includes a polyisoprene material. When inserted within the tissue tract “T”, portal member 102 is adapted to establish a substantial seal within the tract “T”, i.e., with the tissue surfaces defining the tract “T”. During insertion, portal member 102 may be compressed to a compressed condition to permit at least partial passage through the tract “T”. Once within the tract “T”, portal member 102 will return toward the normal expanded condition with the outer wall 112 of the portal member 102 establishing a seal with the tissue defining the tissue tract “T”. Portal member 102 may include an insufflation conduit 114 mounted within one of passageways 106 and connectable to a source of insufflation gases to permit passage of gases, CO2, to maintain the pneumoperitoneum. Suitable portal members 102 are disclosed in commonly assigned U.S. patent application Ser. No. 12/244,024, filed Oct. 2, 2008, the entire contents of which is hereby incorporated by reference herein.

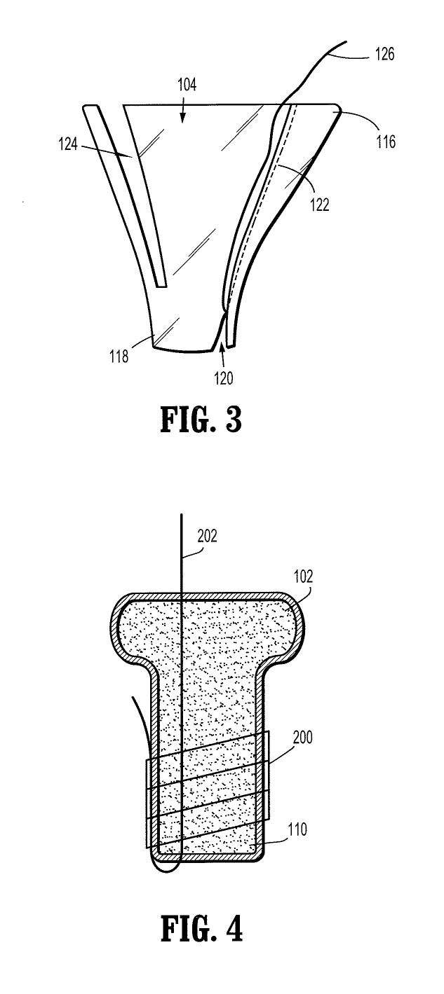

With reference to FIGS. 1 and 3, introducer member 104 is adapted to facilitate insertion of portal member 102 within the tissue tract “T”. Introducer member 104 is substantially elongated defining longitudinal axis “m” and trailing or proximal end 116 and leading or distal end 118. Introducer member 102 defines a longitudinal introducer channel 120 for reception and passage of portal member 102. In one embodiment, introducer member 102 defines a generally tapered or funnel shaped configuration having an internal dimension which decreases from proximal to distal. The relatively narrow configuration of leading or distal end 118 facilitates insertion within tissue tract “T”. Introducer member 104 may be made of any type of suitable material, for example, but not limited to, a polymeric material, and may be rigid or flexible.

Introducer member 104 includes a frangible tear segment or line 122 extending along the wall of the introducer member 102. Frangible tear segment 122 may include a perforated or score line or may incorporate a weakened section in the wall of introducer member 104. Introducer member 104 is adapted to tear along the tear line 122 to permit removal of the introducer member 104 subsequent to insertion of portal member 102 within the tissue tract “T”. Frangible tear segment 122 is oriented along a wall of the introducer member 104 between the proximal end 116 and the distal leading end 118. Introducer member 104 may further include slit 124 that is defined along the wall of the introducer member 104. Slit 124 is dimensioned to permit insufflation conduit 114 of the portal member 102 to pass therethrough as the portal member 102 is advanced through introducer member 104.

A tether 126 may be attached adjacent frangible segment or line 122 and extending toward distal leading end 118 of introducer member 104. Tether 126 may be made of a material, such as, wire, suture, or shape-memory alloy. Tether 126 is adapted to separate the wall of the introducer 104 along the frangible tear line 122 as the tether 126 is selectively pulled in a proximal trailing direction.

In embodiments of the present disclosure, surgical portal and introducer 100 may come preassembled with portal member 102 disposed within introducer member 104. In the alternative, portal member 102 may be positioned within introducer member 104 at the surgical theatre or site.

A method of introducing and deploying portal member 102 includes positioning leading or distal end 118 of introducer member 104 within the tissue tract “T” and advancing the leading end 118 to a predetermined depth. It is envisioned that the introducer member 104 may be made from a translucent-type material such that the clinician may monitor the depth the introducer/portal combination is being deployed within a tissue tract “T”. Thereafter, portal member 102 is positioned within proximal or trailing end 116 of introducer member 104 (if not preassembled as hereinabove discussed). Upon insertion, portal member 102 compresses to fit within the inner boundary of introducer channel 120 of introducer member 104. Portal member 102 is advanced relative to tissue tract “T” by either advancing the portal member 102 within introducer member 104 or advancing the introducer member 104 further into the tract “T”. During advancement, insufflation conduit 114 of portal member 102 may traverse slit 124 in the wall of introducer member 104. Once portal member 102 is located within the tissue tract “T”, e.g., with leading and trailing ends 118, 116 of the portal member 102 on opposed sides of the body wall (e.g., the abdominal cavity wall), introducer member 104 is removed by pulling tether in a general proximal direction or radial outward direction relative to longitudinal axis “m” to effect tearing of the introducer member 104 along frangible segment 122. Introducer member 104 is removed and portal member 102 expands toward its normal expanded condition in sealed engagement with the tissue defining the tissue tract.

In another embodiment illustrated in FIG. 4, a portal member 102 is shown wrapped with a suitable band material 200, for example, but not limited to, a tape material or a thin plastic material. Band material 200 may be kept intact by an adhesive and/or hook and loop fastener, such as VELCRO. At least a length (or, possibly, the entire length) of portal member 102 may be wrapped by the band material 200 such that portal member 102 is compressed. After portal member 102 is wrapped and compressed by band material 200, the portal member 102 is inserted into a tissue tract “T” of a patient at a desired depth. It is envisioned that a thin tether 202, e.g., wire or suture, is disposed within the band material 200. In this configuration, when the tether 202 is pulled by a clinician, it results in cutting and tearing the band material 200 to release portal member 102 to permit the portal member 102 to assume or move toward an expanded state. In embodiments, band material 200 may be perforated.

While several embodiments of the disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical portal and introducer assembly, which comprises:
an introducer dimensioned for at least partial positioning within a tissue tract, the introducer defining a longitudinal axis and having a longitudinal introducer channel extending therethrough, the introducer defining leading and trailing ends, the introducer includes a frangible segment adapted to separate to expose the introducer channel; and
a portal positionable within the longitudinal channel of the introducer, the portal having at least one longitudinal port for passage of a surgical object, the portal comprising a compressible material and being adapted to transition from a first expanded condition to a second compressed condition upon advancement through the longitudinal channel of the introducer to facilitate passage through the introducer whereby, upon separating of the frangible segment, the portal is released from the longitudinal channel to transition toward the first expanded condition to be generally secured within the tissue tract.

2. The surgical assembly according to claim 1 wherein the frangible segment includes a tear line defined along the introducer.

3. The surgical assembly according to claim 2 including a tether secured to the introducer adjacent the tear line, the tether being manipulable to cause tearing along the tear line.

4. The surgical assembly according to claim 2 wherein the introducer defines a generally tapered configuration where an internal dimension of the longitudinal introducer channel generally decreases from the trailing end thereof to the leading end thereof.

5. The surgical assembly according to claim 1 wherein the portal defines leading and trailing ends, the at least one longitudinal port extending between the leading and trailing ends and being adapted for reception of an object whereby compressible material defining the at least one port is adapted to deform to establish a substantial sealed relation with the object.

6. The surgical assembly according to claim 5 wherein the portal includes a plurality of longitudinal ports.

7. The surgical assembly according to claim 1 wherein the portal comprises one of a foam material or a gel material.
